(19) United States
(12) Patent Application Publication
(10) Pub. No.: US 2004/0243017 A1
(43) Pub. Date: Dec. 2, 2004

(54) Anesthesia and sedation monitoring system and method

(76) Inventor: Elvir Causevic, Ellisville, MO (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 10/485,750

(22) PCT Filed: May 6, 2003
(86) PCT No.: PCT/US03/14168

(51) Int. Cl. A61B 5/04; A61B 5/00

(52) U.S. Cl. 600/544; 600/559

(57) ABSTRACT

A method for monitoring the depth of anesthesia experienced by a patient includes identifying a change in one or more evoked bio-potentials (100) using a wavelet transform, and calculating at least one index (108) indicative of the depth of anesthesia experienced by the patient based on the changes in the evoked bio-potentials (100) over a period of time during which anesthesia is administered to the patient. Optionally, changes in random electroencephalogram activity, pulse oximetry measurements, and blood gas measurements are combined with the changes in the evoked bio-potentials (100) in calculations of the index. The resulting indices are optionally displayed in a graphical representation (110) of the level of anesthesia experienced by the patient.

### TECHNICAL FIELD

The present invention relates generally to medical monitoring systems utilized to monitor the vital statistics of a patient, and more particularly to a system and method for monitoring the brain activity of a patient under sedation or anesthesia.

### BACKGROUND ART

In the medical field of anesthesiology, patients must be carefully and continuously monitored to achieve an appropriate balance between delivery of too much and too little anesthetic or sedative. Delivery of an inadequate amount of anesthetic results in patient awareness during the procedure or recall, while excessive amounts of anesthetic or sedative risk central nervous system damage from ischemia due to inadequate perfusion. In recent years, the critical importance of depth-of-anesthesia or sedation monitoring has been highlighted by highly publicized incidents of patients' recall of, or sensation awareness during surgery, and incidents of serious injury or death resulting from delivery of excessive amounts of anesthetic. Most anesthesia-related malpractice suits are premised on inadequate monitoring.

More specifically, known cerebral hemodynamic monitoring techniques include pulse oximetry and infrared spectroscopy, which measure cerebral oxygen saturation. Transcranial Doppler sonography is a noninvasive technique providing real-time continuous measurements of blood flow velocity and other hemodynamic parameters such as direction of blood flow and pulsatility in major intracranial vessels. These continuous measurements are utilized as indicators of the status of collateral cerebral circulation, and provide early indications of any disruption of cerebral perfusion which could result in cases of brain ischemia or death.

Electrophysiological monitoring techniques include the use of the electroencephalogram (EEG), such as is described in U.S. Pat. No. 5,287,859 to John, U.S. Pat. No. 6,052,619 to John, and U.S. Pat. No. 6,385,486 to John et al. At lower levels of sedation or anesthesia, the degree of randomness of the cortical EEG signal is correlated with the level of awareness of the patient, and EEG activity is used as an indicator of approaching alertness in a patient. In particular, EEG monitoring alone is not an adequate indicator of deep, possibly excessive sedation or anesthesia, which can lead to reduced function of the midbrain or the brainstem. In addition, a cortical EEG recording is non-repetitive, typically noisy, susceptible to signal artifacts, and can be difficult to interpret for the purposes of anesthesia or sedation monitoring.

Another known monitoring technique is based on monitoring specific evoked potentials in a selected sensory pathway, such as the auditory pathway. Such a technique is typically employed when certain neural structures in specific sensory pathways are known or believed to be at risk of damage. A sensory stimulus is introduced, and the resulting neural activity generates a wave pattern that is analyzed. The technique relies on adequate discrimination of waveforms using parameters such as peak latency and peak amplitude. Real time changes of the parameters provide a basis for calculating the speed of electrical conduction at the sensory pathway from the peripheral receptor to the sensory cortex. However, evoked signals are intermixed with random EEG activity. To obtain an adequate signal, most hospitals must resort to complex recording set-ups with custom designed monitoring equipment to eliminate or reduce noise in the inauspicious electrical recording environment of an operating room. To adequately discriminate evoked potentials from random activity, computer averaging techniques are employed.

The complex auditory evoked potential (AEP) is produced upon presentation of an auditory stimulus or series of stimuli, such as a click or a tone burst. The AEP consist of early, middle, and late components.

The early or short latency component of the AEP, the auditory brainstem response (ABR) occurs up to 15 ms after the presentation of the auditory stimulus and is widely used for clinical evaluation of hearing in infants and other individuals who are unable to effectively communicate whether a sound was perceived. In individuals with normal hearing, the ABR generates a characteristic neural waveform. Auditory testing using the ABR typically involves a visual or statistical comparison of a tested individual's waveform to a normal template waveform. Like other evoked potentials, the ABR is recorded from surface electrodes on the scalp. However, the electrodes also record the background noise comprised of unwanted bio-potentials resulting from other neural activity, muscle activity, and nonphysiological sources in the environment.

The middle component of the AEP, the auditory mid-latency response (AMLR), also referred to as the middle latency auditory evoked potential (MLAEP) occurs 15 ms-100 ms after the presentation of the auditory stimulus, and is believed to reflect primary, non-cognitive cortical processing of auditory stimuli. Lately, the AMLR or MLAEP has been of particular interest as a measure of depth of anesthesia.

It is known that the AMLR consists of positive and negative waves that are sensitive to sedatives and anesthetics. In general, increasing the level of sedation or anesthetic increases the latency of these waves, and simultaneously decreases the amplitude. For monitoring purposes, changes in the AMLR waves are quantified as latency to peak, amplitude, and rate of change, and are sometimes combined in a single index.

Alternatively, it is known that a 40 Hz auditory signal can induce an enhanced “steady-state” AEP signal. Conventional signal averaging over a period of time is required to extract the AMLR signal from background EEG signals, but adequate signals usually may be obtainable in about 30-40 seconds. The existence of an intact AMLR is believed to be a highly specific indicator for the awake state of a patient, and gradual changes in the depth of sedation or anesthesia appear to be reflected by corresponding gradual changes in the AMLR.

Another component of the complex AEP, the auditory late response (ALR) is believed to be especially sensitive to the level of sedation or anesthesia applied to a patient, and exhibits a distinct flattening of the waveform at a relatively light level of sedation or anesthesia, among other features.

The AEPs are characterized as a “weak bio-signals” which presents a significant technical problem in analyzing and using the AEP, especially when speed and accuracy are critical. Signal processing using linear averaging techniques, filtering, or conventional denoising is known. However, these techniques remain especially limited in ability to process weak biosignals rapidly and, in some cases, accurately.

Ideally, a brain activity monitoring technique is needed which is sufficiently sensitive to provide a near instantaneous indicator of small functional changes in the brain of a patient permitting immediate corrective measures to be taken in ample time before recall, awareness, or tissue damage becomes an issue. However, known anesthetic monitoring techniques, including those that focus on measures of cerebral perfusion or electrophysiologic function in the brain, are limited in terms of sensitivity and speed, and thus the ability to anticipate and allow timely response to significant functional changes. Against this background, a need exists for improved methods and systems for monitoring the brain function and depth of sedation or anesthesia in a patient.

### SUMMARY OF THE INVENTION

A method of the present invention for monitoring the depth of sedation or anesthesia of a patient includes the steps of providing the patient with a repetitive audio stimulus, obtaining signal data representative of an auditory evoked potential, including an auditory brainstem response, over a period of time, and calculating an index indicative of the depth of sedation or anesthesia utilizing an observed change in the AEP over the period of time.

In an alternate embodiment, a method of the present invention for monitoring the depth of sedation or anesthesia of a patient includes the steps of providing the patient with a repetitive audio stimulus, obtaining signal data representative of a an auditory evoked potential, including an auditory brainstem response (ABR), a auditory mid-latency response (AMLR), and an auditory latency response (ALR) over a period of time, and calculating a single index indicative of the depth of sedation or anesthesia utilizing observed changes in the ABR, AMLR, and ALR over the period of time, and/or individual indices.

In a next alternate embodiment, a method of the present invention for monitoring the depth of sedation or anesthesia in a patient includes the steps of obtaining signal data corresponding to at least one evoked bio-potential over a period of time, determining a change in the signal data over the period of time, and calculating at least one index indicative of the depth of sedation or anesthesia in the patient utilizing observed changes in the signal data over the period of time.

In a next alternate embodiment, a method of the present invention for monitoring the depth of sedation or anesthesia in a patient includes the steps of obtaining signal data corresponding to at least one evoked bio-potential over a period of time, the at least one evoked bio-potential selected from a set including auditory evoked bio-potentials, evoked electroencephalogram bio-potentials, evoked somatosensory bio-potentials (SEP), and evoked visual bio-potentials (VEP), determining a change in the signal data over the period of time, and calculating at least one combined or single index indicative of the depth of anesthesia in the patient utilizing observed changes in the signal data over the period of time.

In a next alternate embodiment, a method of the present invention for monitoring the depth of sedation or anesthesia in a patient includes the steps of obtaining signal data corresponding to at least one evoked bio-potential over a period of time, the at least one evoked bio-potential selected from a set including auditory evoked bio-potentials, evoked electroencephalogram bio-potentials, evoked somatosensory bio-potentials, and evoked visual bio-potentials, determining a change in the signal data over the period of time, and calculating at least one combined index indicative of the depth of sedation or anesthesia in the patient utilizing observed changes in the signal data over the period of time together with one or more a pulse oximetry measurements.

In a next alternate embodiment, a method of the present invention for monitoring the depth of sedation or anesthesia in a patient includes the steps of obtaining signal data corresponding to at least one evoked bio-potential over a period of time, the at least one evoked bio-potential selected from a set including auditory evoked bio-potentials, evoked electroencephalogram bio-potentials, evoked somatosensory bio-potentials, and evoked visual bio-potentials, determining a change in the signal data over the period of time, and calculating at least one combined index indicative of the depth of sedation or anesthesia in the patient utilizing observed changes in the signal data over the period of time together with one or more a blood gas measurements and/or breath gas measurements.

In a next alternate embodiment, a method of the present invention provides a basis for generating a visual representation of a patient's brain, in which the level of activity and the depth of sedation or anesthesia for different regions of the patient's brain is graphically represented.

The foregoing and other objects, features, and advantages of the invention as well as presently preferred embodiments thereof will become more apparent from the reading of the following description in connection with the accompanying drawings.

### BRIEF DESCRIPTION OF DRAWINGS

In the accompanying drawings which form part of the specification:

FIG. 1 is a block diagram representation of an apparatus of the present invention;

FIG. 2 is a graphical representation of an auditory stimulus, i.e., tone, presented to a patient;

FIG. 3A is a graphical representation an auditory potential response evoked in a patient with a normal level of awareness in response to the auditory stimulus of FIG. 2;

FIG. 3B is a graphical representation of an auditory potential response evoked in a patient experiencing anesthesia, in response to the auditory stimulus of FIG. 2;

FIG. 4 is a graphical representation of a random EEG activity present in the patient during the period of time represented in the graph in FIG. 3;

FIG. 5 is a block diagrammatic view of a system for monitoring depth of anesthesia in a patient according to the methods of the present invention with optional elements shown in phantom; and

FIG. 6 is a block diagrammatic view of a visual graphic representation of indices representative of depth of anesthesia localized to different regions of the brain of a patient.

Corresponding reference numerals indicate corresponding parts throughout the several figures of the drawings.

### BEST MODE FOR CARRYING OUT THE INVENTION

The following detailed description illustrates the invention by way of example and not by way of limitation. The description clearly enables one skilled in the art to make and use the invention, describes several embodiments, adaptations, variations, alternatives, and uses of the invention, including what is presently believed to be the best mode of carrying out the invention.

As used herein, it is intended that sedation and anesthesia refer to well-known classes of drugs or chemicals which affect the functioning of the nervous system of a patient. The present invention is equally applicable to monitoring the effect of various types of sedatives and anesthetics on a patient. To simplify the following discussion, the terms “anesthetic” and “anesthesia” as well as the phrase “depth-of-anesthesia” will be understood to be interchangeable with “sedative”, “sedation”, and “depth-of-sedation”, respectively, unless otherwise specifically distinguished.

The apparatus and method of the present invention are based in part on the concept that an auditory brain response in a patient is useful as an indicator of depth of sedation or anesthesia in a patient. The methods described herein involve utilizing signal data representative of one or more of a patient's EEG, ABR, AMLR, and ALR bio-potentials, to provide a rapid monitoring of the depth of anesthesia in the patient. Alternative methods of the present invention involve combining signal data representative of one or more evoked bio-potentials in a patient with signal data representative of the brain's activity. These signals may be representative of a random EEG, a SEP, a VEP, the AMLR, or the ALR, and are utilized to provide for further improved monitoring of the depth of anesthesia in the patient.

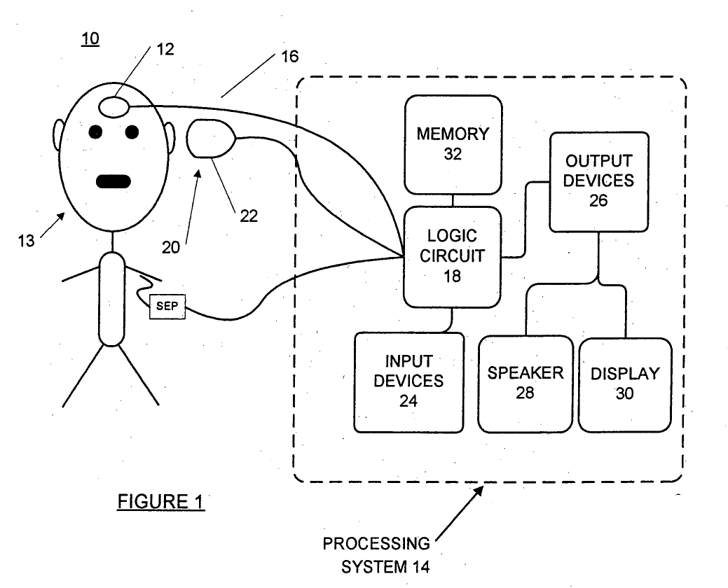

An apparatus of the present invention is shown generally at 10 in FIG. 1. The apparatus 10 includes at least one electrode 12 configured to measure electrical bio-potential signals in a patient 13, such as that shown in co-pending WO Patent Application No. US03/03881 for “Apparatus For Evoking And Recording Bio-potentials”, herein incorporated by reference. The at least one electrode 12 is operatively coupled to a processing system 14 via a lead line 16. Within the processing system 14, a logic circuit 18, such as a micro-processor, micro-controller, or general purpose computer is configured to receive data signals from the at least one electrode 12. The logic circuit 18 is configured to control at least one patient stimulator 20 to provide a controlled stimulus to the patient 13. The stimulator 20 consists of a speaker 22 configured to present a click, tone, or other discrete audio stimulus to an ear of the patient 13. Preferably, a series of clicks, tones, or other discrete or continuous audio stimulus is provided to the ear of the patient 13, generating a series of responses. A suitable processing system 14 is that shown in co-pending U.S. patent application Ser. No. 10/252,345 for a “Handheld Low Voltage Testing Device”, herein incorporated by reference. Those of ordinary skill in the art will recognize that the processing system 14 of the present invention is not limited to providing only discrete audio stimulus, and may be configured to provide visual, tactile, olfactory, or gustatory stimulus to the patient 13.

Preferably, the processing system 14 is further configured with one or more conventional operator inputs 24, such as buttons or switches, and one or more conventional outputs 26, such as a speaker 28 or visual display device 30. Memory or data storage components 32 associated with the processing system 14 are configured to store at least operating instructions for the logic circuit 18 and signal data received from the at least one electrode 12. When executed by the processing system 14, the stored operating instructions for the logic circuit 18 configure the logic circuit 18 to carry out the method of the present invention as set forth herein, including the basic steps of providing a stimulus to a patient, observing and monitoring resulting evoked bio-potential signals in the patient 13, optionally denoising the received evoked bio-potential signals, calculating signal features, and either generating an index of patient awareness for display to an operator or providing a representation of patient neural activity.

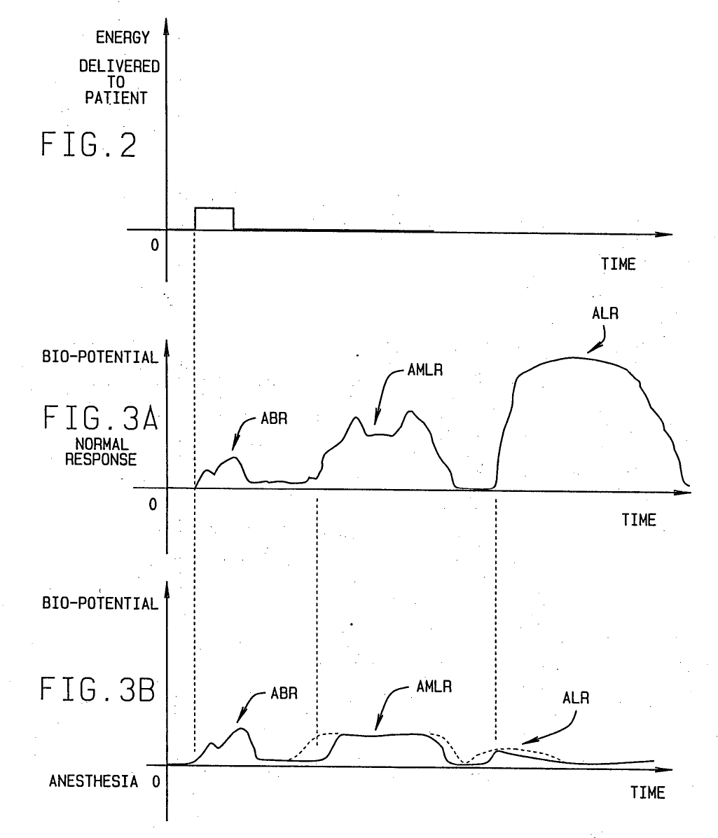

In a preferred embodiment, the method of the present invention requires presenting a stimulus to the patient 13 using the stimulator 20. The stimulus is preferably a predetermined auditory stimulus, i.e., a tone burst such as represented in FIG. 2, or series of clicks, and is presented over a period of time, such as during the administration of anesthesia. The presentation of the auditory stimulus evokes one or more bio-potential responses in the nervous system of the patient, such as the complex auditory evoked potential as shown in FIG. 3A. The complex auditory evoked potentials shown in FIG. 3A include at least three distinct components, the auditory brainstem response, the auditory middle latency response, and the auditory late response. Components of the AEPs and other evoked bio-potentials which are generated in response to stimuli are known to change in response to the depth of anesthesia which is experienced by the patient, such as shown in FIG. 3B. These changes may be reflected in a reduction in the amplitude of the observed evoked bio-potential components, or a change in the response time.

Accordingly, signal data representative of the one or more evoked bio-potential responses in the patient, including the complex auditory evoked potentials, is obtained by the at least one electrode 12 and monitored by the processing system 14 during the administration of anesthesia to the patient 13. The signal data may represent the response to a single stimulus, or may be a representative or average response from a series of stimulus presented to the patient over a short period of time. The obtained signal data representative of the complex auditory evoked potential is processed by the processing system 14 to identify changes in the auditory brainstem response component of the AEP, and optionally, one or more additional evoked bio-potential signals. The changes are, in turn, utilized by the processing system 14 to calculate an index value which is indicative of the level of awareness or depth of anesthesia experienced by the patient 13.

The signal data representative of the one or more complex AEP further includes a components which correspond to the auditory middle latency response in the patient 13. In an alternative method of the present invention, the obtained signal data representative of the complex auditory evoked potential is processed by the processing system 14 to identify changes in the auditory middle latency response component of the AEP. The identified changes are utilized by the processing system 14 together with the identified changes in the ABR for calculating the single representative index value which is indicative of the depth of anesthesia experienced by the patient 13.

The signal data representative of the one or more complex AEP further includes a components which correspond to the auditory late response in the patient 13. In an alternative method of the present invention, the obtained signal data representative of the complex auditory evoked potential is processed by the processing system 14 to identify changes in the auditory late response component of the AEP. The identified changes are utilized by the processing system 14 together with the identified changes in the ABR for calculating the single representative index value which is indicative of the depth of anesthesia experienced by the patient 13.

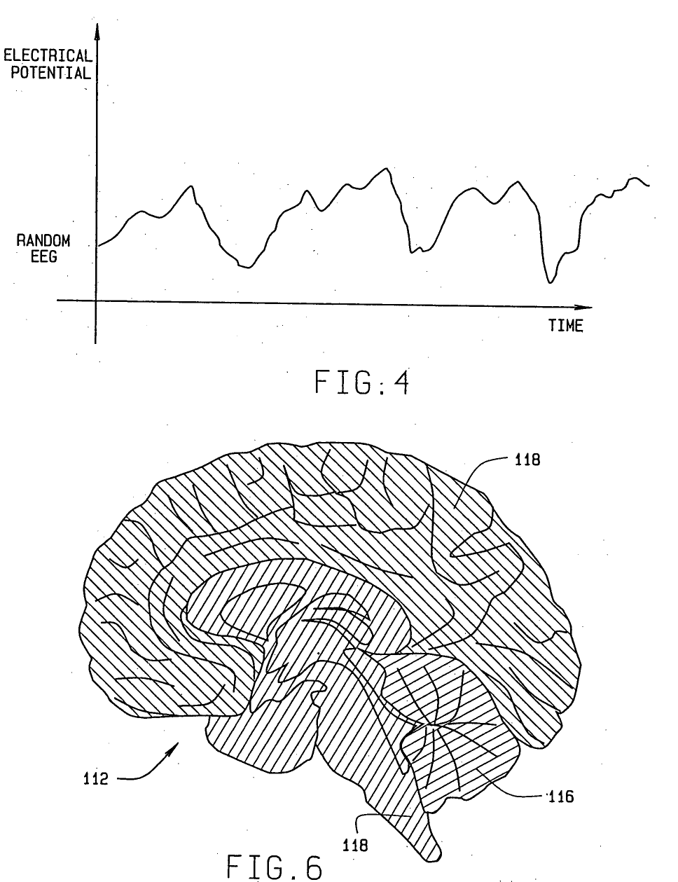

In an alternative method of the present invention, concurrent with the monitoring of the complex AEP signal data, additional signal data representative of the random electroencephalogram activity of the nervous system of the patent, shown in FIG. 4, is obtained from additional electrodes 12 and monitored by the processing system 14 during the administration of anesthesia to the patient 13. Prior to being received at the processing system 14, the obtained electroencephalogram signal data is pre-processed in a conventional manner through a series of frequency band-pass filters and the resulting discrete EEG frequency bands routed to separate channels for input to the processing system 14.

It is known that the ratio of energies in different frequencies of the EEG signals data are indicators of patient awareness. The filtered EEG frequency bands on each channel are processed and characterized by the processing system 14 in a conventional manner for EEG signal data, to provide a representative waveform for each EEG output channel. Each of the EEG representative waveforms are monitored to identify any variations over time, which in turn, are utilized together with the identified changes in the monitored components of the complex AEP for calculating the single representative index value which is indicative of the depth of anesthesia experienced by the patient 13.

Determining changes in the complex AEP signal data or any component thereof, such as the ABR, or determining a change in an EEG waveform over the period of time, requires denoising the signal data. With respect to particularly weak evoked bio-potentials, such as the ABR, conventional denoising techniques do not adequately extract the desired features of the data signal with sufficient clarity and speed to be useful for calculating an index which is representative of the real-time depth of anesthesia experienced by a patient.

The apparatus and methods of the present invention utilize wavelet transformation of the data signals for the extraction of signal features and the calculation of a depth of anesthesia index. The wavelet transform is an integral transform that projects the original signal onto a set of unconditional basis functions called wavelets. Preferably, the wavelet utilized in the transformation is discrete and either an orthogonal or bi-orthogonal wavelet which has finite support and which may be used with discrete wavelet transforms. However, in alternate embodiments, a series of different wavelets may be utilized for extraction of signal features and the calculation of the depth of anesthesia index, and some of the wavelets in the series may be continuous, and are not limited to orthogonal or bi-orthogonal wavelets. The wavelet transform is carried out on the data signal to obtain a number of wavelet coefficients at different scales.

Optionally, the wavelet transformation is further utilized to perform an optional signal denoising operation prior to the extraction of the signal features and the calculation of the depth of anesthesia index. Optional denoising of the data signals is accomplished by using wavelet coefficient thresholding to separate incoherent noise from the coherent signals. Specifically, the wavelet transform is carried out on the data signal to obtain a number of wavelet coefficients at different scales. A threshold level is established, and any coefficients which lie below the established threshold, i.e., which correspond to noise components, are set to zero or reduced.

Wavelet transformation of the data signals provides sufficiently fast and adequate denoising and feature extraction of the signal data such that the signal data can be used for rapid feedback in the context of monitoring the patient for the depth of anesthesia. Wavelet transformations do not require large amounts of computer memory, and there facilitate the implementation of the methods of the present invention in small, portable devices, and in handheld anesthesia monitoring devices.

As with the traditional Fourier transform, continuous and discrete versions of wavelet transforms exist, and either may be utilized in the context of the present invention for feature extraction, signal denoising, and the calculation of the depth-of-anesthesia index. Those of ordinary skill in the art will recognize that there are many types of wavelets which may be utilized in developing a wavelet transform, and that there are numerous types of wavelet transforms. Representative examples may be found in U.S. Pat. No. 5,384,725 to Coifman et al. for “Method and Apparatus For Encoding and Decoding Using Wavelet-Packets” and U.S. Pat. No. 5,526,299 to Coifman et al. for “Method and Apparatus For Encoding and Decoding Using Wavelet-Packets” which are herein incorporated by reference. Preferably, the wavelet utilized in the transformation is either an orthogonal or bi-orthogonal wavelet which has finite support and which may be used with discrete wavelet transforms.

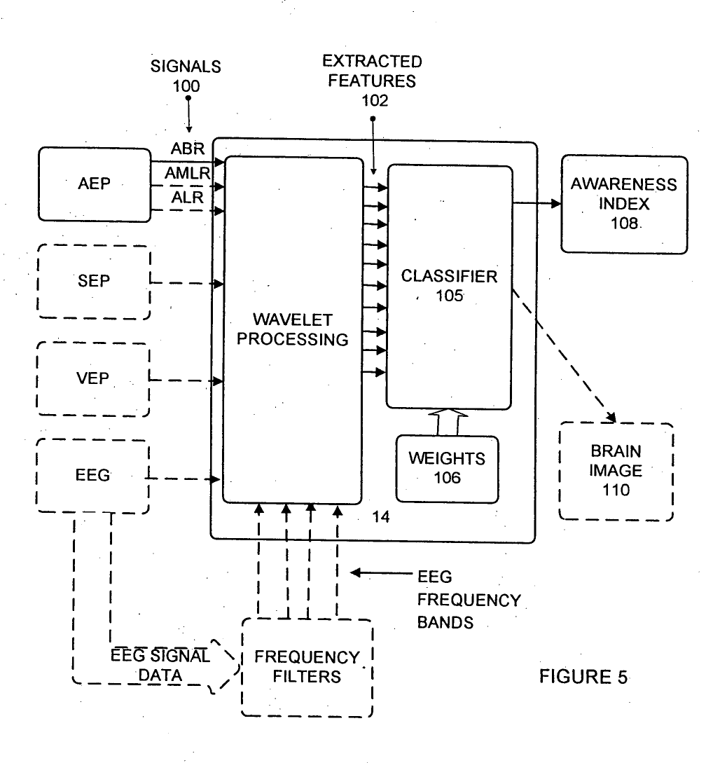

As shown in FIG. 5, to calculate the index which is representative of the real-time depth of anesthesia experienced by a patient, each of the methods of the present invention utilizes the same basic processing methodology on a different set of input data signals. For each method of the present invention, the observed and monitored data signals 100 are processed by the processing system 14 using one or more wavelet transforms to optionally reduce the level of signal noise present, to enhance the signal data corresponding to the observed and monitored bio-potential or random EEG frequency, and for signal feature extraction. The extracted features 102 of the data signals are utilized by the processing system 14 as input to a classifier 104 consisting of a general linear model, discriminant basis, or other classification algorithm wherein predetermined weights 106 are assigned to each processed signal component or extracted feature 102. The predetermined weights assigned to each processed signal component are clinically determined and selected according to the set of input data signals and the characteristics of the patient, i.e., weight, age, gender, type of anesthesia used, etc. The resulting values are combined by the processing system 14 to generate one or more indices 108 which are representative of the real-time depth of anesthesia experienced by a patient.

In addition to calculating an index which is representative of the real-time depth of anesthesia experienced by a patient, an alternate method of the present invention generates a visual display 110 which is representative of the level of neural activity in one or more regions of the brain of a patient. The signal data representative of the one or more evoked bio-potential responses in the patient, such as the complex AEP, the SEP, or the VEP, and the random EEG frequency signal data which is obtained and monitored during the administration of anesthesia to the patient is utilized to provide a graphical representation of the depth of anesthesia experienced by the patient. The graphical representation, shown in FIG. 6, is generated by mapping visual representations of the values of the one or more evoked bio-potential responses or random EEG frequency signals onto a representation 112 of the brain of the patient to provide a graphical representation of the level of activity present therein.

For example, a graphical representation 112 of the brain of the patient is provided which includes first region 114 representative of a brainstem, at least a second region representative of a midbrain 116, and at least a third region representative of a cortex 118. A value of the one or more evoked bio-potential responses or random EEG frequency signals corresponding to activity in the brainstem of the patient, such as the ABR, is mapped onto the first region 114. Similarly, a value of the one or more evoked bio-potential responses or random EEG frequency signals corresponding to activity in the midbrain of the patient, such as the SEP, is mapped onto the second region 116. Finally, a value of the one or more evoked bio-potential responses or random EEG frequency signals corresponding to activity in the cortex of the patient, such as selected random EEG frequencies, is mapped onto the third region 118. To facilitate a visual identification of the different regions and the associated level of activity as indicated by the mapped values, the values may be visually represented as a grayscale or color shading within each region of the image, such as shown in FIG. 6. For example, white or green shades may be utilized to represent normal neural activity (i.e. activity indicative of patient awareness) in a region of the brain, while black or red shades may be utilized to represent a lack or reduction of observed neural activity (i.e. patient experiencing anesthesia) for a region of the brain.

By providing an operator such as an anesthesiologist with such a graphical representation 112 of the level of neural activity in the brain of a patient, and in particular, a patient who is subjected to anesthesia, a rapid assessment of the level of awareness or depth of anesthesia can be made.

Those of ordinary skill in the art will recognize that alternative representations of the neural activity in the brain of a patient, and in particular, a patient who is subjected to anesthesia may be generated from the measured values of the one or more evoked bio-potential responses or random EEG frequency signals corresponding to neural activity in the brain of the patient. For example, an audible signal can be provided to an anesthesiologist which is representative of the level of neural activity or depth of anesthesia. Any of a number of predetermined audio characteristics, such as tone, pitch, or volume, may be changed to correspond to changes in the level of neural activity or depth of anesthesia of the patient.

The present invention can be embodied in-part in the form of computer-implemented processes and apparatuses for practicing those processes. The present invention can also be embodied in-part in the form of computer program code containing instructions embodied in tangible media, such as floppy diskettes, CD-ROMs, hard drives, flash memory, or an other computer readable storage medium, wherein, when the computer program code is loaded into, and executed by, an electronic device such as a computer, micro-processor or logic circuit, the device becomes an apparatus for practicing the invention.

The present invention can also be embodied in-part in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented in a general-purpose microprocessor, the computer program code segments configure the microprocessor to create specific logic circuits.

In view of the above, it will be seen that the several objects of the invention are achieved and other advantageous results are obtained. As various changes could be made in the above constructions without departing from the scope of the invention, it is intended that all matter contained in the above description or shown in the accompanying drawings shall be interpreted as illustrative and not in a limiting sense.

1. A method for determining the depth of anesthesia experienced by a patient, comprising the steps of:
stimulating the patient with a repetitive stimulus;
obtaining signal data representative of a series of evoked potential waveforms generated by the patient in response to said stimulus;
extracting at least one signal feature from said obtained signal data with at least one wavelet transform; and
calculating a representation of the depth of anesthesia experienced by the patient from said at least one extracted signal feature.

2. The method of claim 1 wherein said step of calculating said representation includes determining a change in said at least one extracted signal feature representative of said series of evoked potential waveforms over said period of time.

3. The method of claim 1 wherein said repetitive stimulus is an audio stimulus, and said of evoked potential waveforms are auditory evoked potential waveforms.

4. The method of claim 3 wherein said obtained signal data is representative of said series of auditory evoked potentials includes signal data representative of at least one auditory brainstem response; and
wherein said step of calculating said representation includes determining a change in said auditory brainstem response over said period of time.

5. The method of claim 3 wherein said obtained signal data is representative of said series of auditory evoked potentials includes signal data representative of at least one auditory middle latency response; and
wherein said step of calculating said representation includes determining a change in said auditory middle latency response over said period of time.

6. The method of claim 3 wherein said obtained signal data is representative of said series of auditory evoked potentials includes signal data representative of at least one auditory late response; and
wherein said step of calculating said representation includes determining a change in said auditory late response over a period of time.

7. The method of claim 1 further including the step of obtaining additional signal data representative of random electroencephalogram activity in the patient over said period of time; and
wherein said step of calculating a representation of the depth of anesthesia experienced by the patient from said obtained signal data further utilizes said obtained additional signal data.

8. The method of claim 7 wherein said signal data representative of said random electroencephalogram activity comprises a series of waveforms, and
wherein utilizing said obtained additional signal data includes:
(a) determining a change in said random electroencephalogram activity over said period of time; and
(b) denoising said random electroencephalogram activity signal data with at least one wavelet transform.

9. The method of claim 1 wherein said at least one wavelet transform comprises a discrete wavelet transform.

10. The method of claim 1 wherein said at least one wavelet transform comprises a continuous wavelet transform.

11. The method of claim 1 further including the step of:
obtaining at least one additional physiological measurement from the patient, said at least one additional physiological measurement selected from the set of blood gas measurements and breath gas measurements;
calculating a representation of the depth of anesthesia experienced by the patient from said at least one extracted signal feature and said at least one additional physiological measurement.

12. The method of claim 1 further including the step of denoising said obtained signal data with at least one wavelet transform prior to extracting at least one signal feature from said obtained signal data.

13. The method of claim 1 further including the step of selecting an orthogonal wavelet for said wavelet transform.

14. The method of claim 1 further including the step of selecting an bi-orthogonal wavelet for said wavelet transform.

15. The method of claim 1 wherein the step of extracting at least one signal feature from said obtained signal data with at least one wavelet transform includes calculating at least one wavelet coefficient.

16. A method for monitoring the depth of anesthesia in a patient, comprising the steps of:
obtaining signal data corresponding to a series of auditory evoked potentials in the patient over a period of time;
denoising said obtained signal data with at least one wavelet transform;
identifying at least one change in said obtained signal data over said period of time;
obtaining additional signal data corresponding to random electroencephalogram activity in the patent during said period of time;
identifying at least one change in said additional signal data over said period of time; and
calculating at least one index indicative of the depth of anesthesia experienced by the patient utilizing said identified changes in said signal data and said identified changes in said additional signal data over said period of time.

17. The method of claim 16 wherein said signal data includes data representative of a auditory brainstem response in the patient; and
wherein the step of identifying at least one change in said signal data includes identifying a change in said data representative of said auditory brainstem response.

18. The method of claim 16 wherein said signal data includes data representative of a auditory middle latency response in the patient; and
wherein the step of identifying at least one change in said signal data includes identifying a change in said data representative of said auditory middle latency response.

19. The method of claim 16 wherein said signal data includes data representative of a auditory late response in the patient; and
wherein the step of identifying at least one change in said signal data includes identifying a change in said data representative of said auditory late response.

20. The method of claim 16 wherein said signal data includes signal data corresponding to an auditory brainstem response in the patient, signal data corresponding to an auditory middle latency response in the patient, and signal data corresponding to an auditory late response in the patient;
wherein the step of identifying at least one change in said signal data includes identifying a change in said auditory brainstem response signal data; and
wherein the step of calculating said at least one index includes utilizing said identified change in said auditory brainstem response signal data to calculate an index indicative of the depth of anesthesia in the brain of the patient.

21. The method of claim 20 wherein the step of identifying at least one change in said signal data includes identifying a change in said auditory middle latency response signal data; and
wherein the step of calculating said at least one index includes utilizing said identified change in said auditory middle latency response signal data to calculate a second index indicative of the depth of anesthesia in the brain of the patient.

22. The method of claim 21 wherein the step of identifying at least one change in said signal data includes identifying a change in said auditory late response signal data; and
wherein the step of calculating said at least one index includes utilizing said identified change in said auditory late response signal data to calculate a third index indicative of the depth of anesthesia in the brain of the patient.

23. The method of claim 22 further including the steps of:
providing a graphical representation of the depth of anesthesia experienced by the patient; and
mapping the values of the first, second, and third indices of the depth of anesthesia in the brain of the patient onto said graphical representation.

24. The method of claim 23 wherein the step of providing said graphic representation includes providing a graphical representation of a brain having at least a first region representative of a brainstem, at least a second region representative of a midbrain, and at least a third region representative of a cortex;
wherein a value representative of said first index is mapped onto said first region;
wherein a value representative of said second index is mapped onto said second region; and
wherein a value representative of said third index is mapped onto said third region.

25. The method of claim 24 wherein each of said mapped values is a visually distinct shade of gray.

26. The method of claim 24 wherein each of said mapped values is a visually distinct color.

27. The method of claim 16 wherein identifying at least one change in said signal data includes denoising said signal data with at least one wavelet transform.

28. The method of claim 27 wherein identifying at least one change in said signal data includes denoising said signal data with at least one discrete wavelet transform.

29. The method of claim 16 wherein identifying at least one change in said additional signal data includes denoising said additional signal data with at least one wavelet transform.

30. The method of claim 29 wherein identifying at least one change in said additional signal data includes denoising said additional signal data with at least one discrete wavelet transform.

31. The method of claim 16 wherein the step of calculating said at least one index indicative of the depth of anesthesia experienced by the patient includes analyzing said change in said signal data and analyzing said change in said additional signal data to obtain a single index indicative of the depth of experienced by the patient.

32. A method for monitoring neural activity in a patient, comprising the steps of monitoring one or more bio-potential signals evoked in the patient over a period of time, extracting features from said monitored bio-potential signals with at least one wavelet transform; and observing changes in said features extracted from said bio-potential signals responsive to at east one repetitive stimulus over said period of time.

33. The method of claim 32 further including the step of denoising said monitored bio-potential signals with at least one wavelet transform prior to extracting said features.

34. The method of claim 32 wherein said repetitive stimulus is an auditory stimulus, and wherein said observed changes are changes in an auditory brainstem response.

35. The method of claim 34 wherein said observed changes further include changes in an auditory middle latency response.

36. The method of claim 34 wherein said observed changes further include changes in an auditory late response.

37. The method of claim 32 further including the step of monitoring random electroencephalogram activity of the patient for changes over said period of time.

38. The method of claim 32 further including the steps of:
obtaining at least one pulse oximetry measurement from the patient during said period of time; and
utilizing said changes in said bio-potential together with said at least one pulse oximetry measurement to generate a representation of the depth of anesthesia experienced by the patient.

39. The method of claim 32 further including the steps of:
obtaining at least one blood gas measurement from the patient during said period of time; and
utilizing said observed changes together with said at least one blood gas measurement to generate a representation of neural activity of the patient.

40. The method of claim 32 further including the steps of:
obtaining at least one breath gas measurement from the patient during said period of time; and
utilizing said observed changes together with said at least one breath gas measurement to generate a representation of neural activity of the patient.

41. The method of claim 40 wherein said at least one breath gas measurement is a CO₂ measurement.

42. The method of claim 32 further including the steps of:
obtaining at least one blood gas measurement from the patient during said period of time;
obtaining at least one breath gas measurement from the patient during said period of time; and
utilizing said observed changes together with said at least one breath gas measurement, and said at least one breath gas measurement, to generate a representation of neural activity of the patient.

43. The method of claim 32 further including the step of providing a graphical representation of the neural activity of the patient based on said observed changes.

44. The method of claim 43 wherein the step of providing said graphic representation includes providing a graphical representation of a brain having at least a first region representative of a brainstem, at least a second region representative of a midbrain, and at least a third region representative of a cortex; and
mapping said observed changes to at least one associated region.

45. The method of claim 44 wherein each of said mapped observed changes are represented with a visually distinct shade of gray.

46. The method of claim 44 wherein each of said mapped observed changes are represented with a visually distinct color.
